(19) United States
(12) Patent Application Publication
(10) Pub. No.: US 2020/0193867 A1
(43) Pub. Date: Jun. 18, 2020

(54) ELECTRIC APPARATUS FOR ORAL HYGIENE

(71) Applicant: RICHARD DAVID DAVIS, MITZPE RAMON (IL)

(72) Inventor: RICHARD DAVID DAVIS, MITZPE RAMON (IL)

(21) Appl. No.: 16/693,401

(22) Filed: Nov. 25, 2019

Related U.S. Application Data
(60) Provisional application No. 62/780,342, filed on Dec. 17, 2018.

(51) Int. Cl. G09B 19/00; A61C 17/22; A61C 17/26; A61B 1/247

(52) U.S. Cl. CPC G09B 19/0084; A61C 17/221; A61C 2203/00; A61C 17/26; A61B 1/247; A61C 17/224

(57) ABSTRACT

An electric toothbrush assembly including a brush head, an elongated member having a proximal end coupled to the brush head and a distal end configured to engage with a docking station, a camera configured to capture image of a user, and a communication interface, wherein the communication interface enables communication between the electric toothbrush assembly and one or more communication devices. The electric toothbrush assembly is configured to send messages to the one or more communication devices via the communication interface when a predetermined condition is satisfied.

### FIELD

Embodiments of the invention relate to an electric apparatus for maintaining oral hygiene. Specifically, the embodiments of invention relate to an electric toothbrush and procedures for maintaining the oral hygiene.

### BACKGROUND

Oral complications are largely preventable by employing a regular and effective brushing habit. It is important to note that the oral tooth decay is five times as common as asthma, and seven times as common as hay fever, according to the Centers for Disease Control and Prevention (CDC). Centers for Disease Control and Prevention says that 42% of children aged 2 to 11 have had cavities in baby teeth and 21% of those aged 6 to 11 have had cavities in permanent teeth.

We're reaching epidemic proportions of tooth decay especially in younger children with 25,800 children admitted to hospitals with tooth decay every year. One of the points made in the new campaign is that early childhood tooth decay is a serious problem that can result in severe pain, infection and tooth loss even in toddlers. Typically, it is because the lazy middle-class parents do not teach their children to brush their teeth. If this continues, children will have no concept of the importance of oral health and hygiene.

Although we have number of electric toothbrushes available in market, there is still a need for a toothbrush assembly that engages and motivates the user to perform effective brushing. Thus, an electric toothbrush device that provides better oral hygiene management is desirable.

### SUMMARY

The invention, in embodiments thereof, provides an electric toothbrush assembly including a brush head, an elongated member, a camera and a communication interface. The elongated member includes a proximal end and a distal end such that the proximal end is configured to couple to the brush head and the distal end is configured to engage with a docking station. The elongated member is configured to switch between an engaged position and a disengaged position, where the elongated member engages with the docking station in the engaged position and is pulled out of the docking station in the disengaged position. Further, the camera is configured to capture image of a user while being able to switch between an enabled state and a disabled state. The communication interface allows communication between the elongated member and one or more communication devices.

In an embodiment of the invention, the image captured by the camera can be shared with one or more communication devices over the communication interface.

In a further embodiment of the invention, a first message is displayed on a user display of a first communication device when the state of elongated member changes from engaged position to disengaged position.

In a still further embodiment of the invention, a second message is sent to a second communication device when a predetermined condition is satisfied.

Additional aspects of the invention will be set forth in part in the description that follows.

It is to be understood that both the foregoing general description and the following detailed description are exemplary and explanatory only and are not restrictive of the invention.

### BRIEF DESCRIPTION OF THE DRAWINGS

The accompanying drawings, which are incorporated in and constitute a part of this specification, illustrate exemplary embodiments of the invention and together with the description, explain the principles of the invention.

FIGS. 1A and 1B are schematic view of an electric toothbrush assembly illustrating the way a docking station is coupled to the electric toothbrush assembly in accordance with an embodiment of the invention;

FIGS. 2A and 2B illustrates the front view and the side view of the docking station and the electric toothbrush assembly, respectively;

FIG. 3 is a side view of the docking station according to an embodiment of invention;

FIG. 4 illustrates an exploded view of the docking station according to an embodiment of invention;

FIG. 5 illustrates an exploded view of the electric toothbrush assembly according to an embodiment of invention;

FIGS. 6A and 6B are schematic view of the electric toothbrush assembly depicting the camera along with its main components according to one embodiment of the invention;

FIG. 7 illustrates the front view of the docking station in an idle state according to an embodiment of the invention;

FIG. 8 illustrates an active state of the docking station when the electric toothbrush assembly is disengaged from it, according to an exemplary embodiment of the invention;

FIG. 9A illustrates a user holding the electric toothbrush assembly;

FIG. 9B illustrates the initiation of a brushing mode on the docking station according to an embodiment of the invention.

FIG. 10 illustrates a schematic view of a camera mode of the docking station, and the manner docking station and electric toothbrush assembly communicates with a remote communication device according to an exemplary embodiment of the invention.

### DETAILED DESCRIPTION

Reference will now be made in detail to embodiments of the invention, examples of which are illustrated in the accompanying drawings. Wherever possible, the same reference numbers will be used throughout the drawings to refer to the same or like parts.

Embodiments of the invention relate to apparatus and associated methods for maintaining oral hygiene. An electric toothbrush assembly is being used to clean the inner surface, outer surface, and chewing surface of teeth to avoid formation of plaque, dental cavity and other oral complications. The electric toothbrush assembly includes a brush head containing bristles, and an elongated member that is removably coupled to the brush head. The other end of the elongated member may rest inside a docking station such that the docking station is configured to electrically charge the electric toothbrush assembly. The elongated member is configured to switch between an engaged position and a disengaged position from the docking station such that the elongated member may rest inside the docking station in the engaged position, whereas the elongated member is pulled out of the docking station in the disengaged position. In one embodiment of the invention, the docking station holds a communication device such as a mobile phone. The communication device can communicate with the electric toothbrush assembly and one or more other communication devices over a communication interface. Further, the communication device displays a first message whenever the elongated member is in the disengaged position. The first message, for example, includes a multimedia game that aid in achieving effective brushing, where the effective brushing is achieved only when a user brushes his teeth to meet a predetermined condition. In an embodiment, the predetermined condition may be achieved when a user brushes his teeth effectively for a predetermined time interval of, for example, at least 2 minutes to avoid formation of an oral complication.

The electric toothbrush assembly also includes a camera disposed along the elongated member. The camera is configured to capture one or more of an image, an audio, and a video in an enabled state. The electric toothbrush assembly further includes a control unit and a memory. In an example, the control unit makes assessment if brushing event satisfies the predetermined condition. If yes, the camera switches to an enabled state, thereby allowing the user to click a selfie image and/or record an audio and/or video. The image captured or recorded audio/video can be stored in the memory of the electric toothbrush assembly. In addition, the user can also send his image/audio/video from the electric toothbrush assembly to the communication device of the docking station. Further, the image/audio/video can also be sent to a remotely located communication device, for example, a second mobile device. In an example, the remotely located communication device is the mobile device of one of the user's parents. The parent may in return share a message that can be displayed on the communication device of the docking station. where the message may include any multimedia message including a thumbnail, for example, ‘thumbs up’.

Those skilled in the art will recognize that the invention may be manifested in a variety of forms other than the specific embodiments described and contemplated herein. Accordingly, departure in form and detail may be made without departing from the scope and spirit of the invention as described herein.

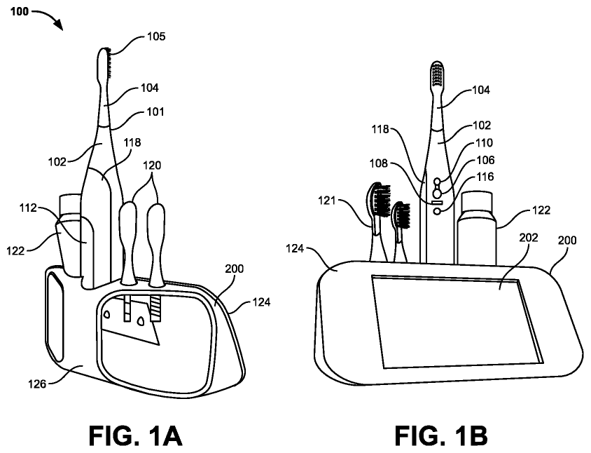

Refer to FIGS. 1A and 1B which illustrate an exemplary embodiment of an electric toothbrush assembly 100 according to the invention.

The embodiment depicts an electric toothbrush assembly 100 in an engaged position with a docking station 200. According to one embodiment, the electric toothbrush assembly 100 may sit inside the docking station 200 in the engaged position. Further, the docking station 200 may electrically charge the electric toothbrush assembly 100 in the engaged position. The charged electric toothbrush assembly 100 can be employed for one or more brushing events to maintain oral hygiene of a user.

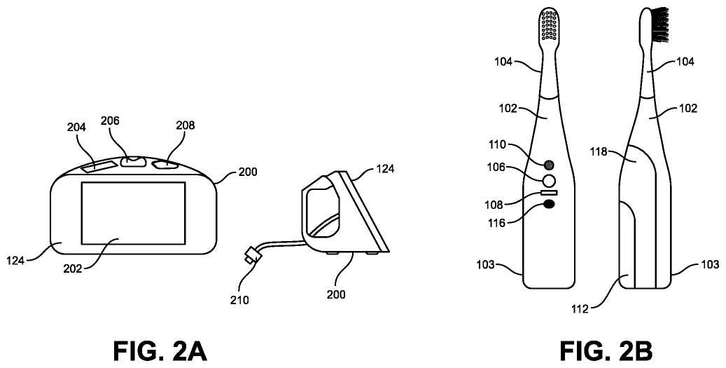

The electric toothbrush assembly 100 includes an elongated member 102 having a proximal end 101 and a distal end 103 (shown in FIG. 2B). While the proximal end 101 of the elongated member 102 is removably coupled to a brush head 104, the distal end 103 (shown in FIG. 2B) may allow the elongated member 102 to engage with the docking station 200. It should be noted that the brush head 104 is rigidly coupled to the elongated member 102 such that any movement of the elongated member 102 is translated to the brush head 104.

Further, the brush head 104 includes a set of bristles 105 extending perpendicularly outwards to the axial length of the brush head 104. The bristles 105 may be composed of any suitable material such as natural polymers, synthetic, a combination thereof, or any other material known in the art sufficient to allow effective brushing. Further, the brush head 104 can be made of a polymer material having sufficient flexibility to traverse through oral cavities and regions. To that end, the brush head 104 can be made of a material capable of allowing movement translation in accordance to the movement of elongated member 102.

The elongated member 102 further includes a camera 106 configured to capture one or more images of a user. The images include a still image, a video image or a combination of both. The camera 106 is disposed along the axial length of the elongated member 102. In FIG. 1B, the camera 106 is shown to be disposed on the elongated member 102 on same side as the bristles 105. However, it should be noted that the camera 106 can be disposed on any side of the elongated member 102. Typically, the camera 106 aims its aperture towards the user such that the user may click one or more images or may record a video while using the electric toothbrush assembly 100. In one embodiment, the camera 106 is used to click a selfie image of the user once he/she successfully completes a brushing event. It should, however, be noted that the camera 106 can be used to capture images of the user at any time during the brushing event. Specifically, the brushing event may be referred to an ‘effective brushing event’ if the user brushes his teeth to meet a predetermined condition. In an exemplary embodiment, the predetermined condition comprises brushing time of a predetermined interval, for example, at least 2 minutes. Alternatively, the predetermined condition may also comprise brushing on all sides of teeth. Further, the predetermined condition may comprise cleaning tongue in addition to teeth. However, it should be noted that the predetermined condition may include any suitable criteria that allows effective brushing to avoid cavities and other oral complications.

In the illustrated embodiment, the camera 106 is detachably coupled to the elongated member 102 of the electric toothbrush assembly 100, but a person skilled in the art may contemplate various other positions where the camera 106 may be located along the length of the electric toothbrush assembly 100. For instance, in an embodiment, the camera 106 may be coupled to the brush head 104. In such an embodiment, the camera 106 can be used to capture images of the oral sections. Further, the camera 106 can couple to the elongated member 102 either removably or permanently.

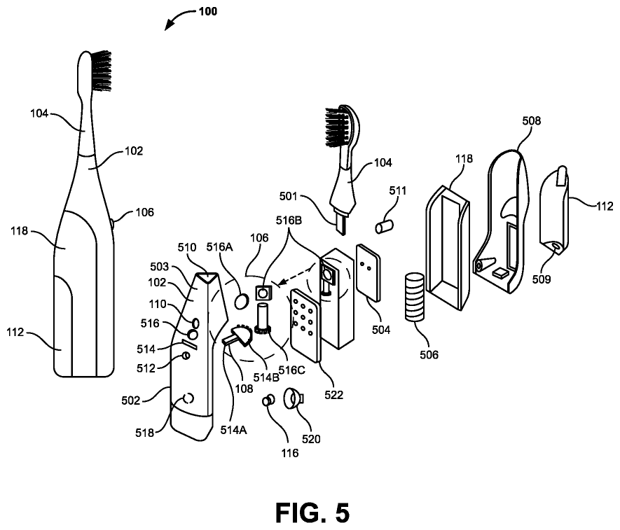

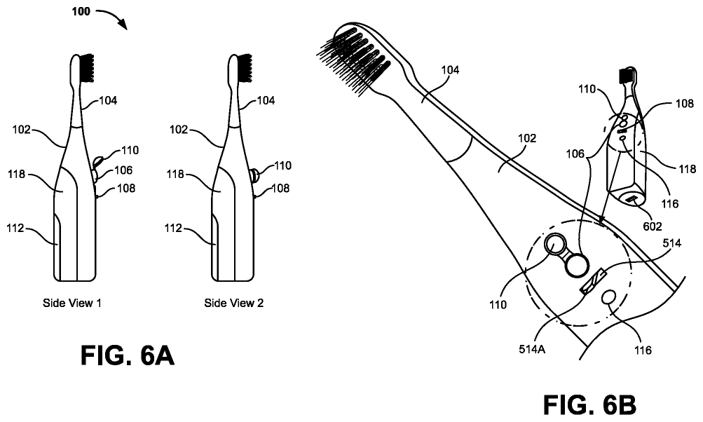

The elongated member 102 further incudes a swiveling member 108 and a camera cover 110. The swiveling member 108 is coupled to the camera 106 and is configured to switch the camera 106 between an enabled state and a disabled state. The camera cover 110 includes a cap that can shift between an up position and a down position. In the down position, the camera cover 110 protects the camera lens (516a, as shown in FIG. 5) from external environment, whereas the user can lift the camera cover 110 to up position so that he/she can use the camera 106 for capturing images/videos. More details on the enabled and disabled states, and the swiveling member 108 are provided in the description of FIG. 5 and FIG. 6 below.

Further, the elongated member 102 includes a battery (506, as shown in FIG. 5) disposed inside the cavity of the elongated member 102. The battery can be protected from external environment using a battery cover 112. The elongated member 102 may also include a charging port (602, as shown in FIG. 6B) that can be disposed along the distal end 103. The charging port may allow the electric charging of the toothbrush assembly 100 while resting inside a cavity (206, shown in FIG. 2A) formed within the docking station 200.

Furthermore, the electric toothbrush assembly 100 includes a power button 116 located along the length of the elongated member 102. Whenever in use, the user holds the electric toothbrush assembly 100 along the length of the elongated member 102 and activates the assembly 100 by pushing the power button 116 ‘ON’. The electric toothbrush assembly 100 also includes a motor (not shown) that helps in oscillation of the bristles 105 whenever the power button 116 is turned ‘ON’. The motor triggers oscillatory movement to the bristles 105 so that the assembly 100 allows cleaning of teeth during one or more brushing events. The user may push the power button 116 ‘OFF’ at any given time to turn off the electric toothbrush assembly 100. The assembly 100 may further include additional component(s) (not shown) to control speed of the motor and/or other functional aspects without limiting the scope of invention.

The docking station 200 further includes a plurality of cavities (204, 206, 208, as shown in FIG. 2A) to receive other auxiliary devices such as extra set of brush heads and/or toothpaste. It should be noted that the plurality of cavities can be used to receive any additional auxiliary device known in the art. For instance, one of the plurality of cavities may receive an additional electric toothbrush assembly, a tongue cleaner, etc. Further, the docking station 200 may include any suitable number of additional set of cavities that can hold extra set of electric toothbrush assembly, brush head, toothpaste and the like.

The electric toothbrush assembly 100 further includes a plurality of flashlights 118 that are disposed along the body of the electric toothbrush assembly 100. In an alternative embodiment, the flashlights 118, can be disposed on all body of the elongated member 102 in a strip manner. In yet another embodiment, the flashlights 118 can also be disposed on the brush head 104. In the preferred embodiment, the flashlights 118 include a set of LED lights. The flashlights 118 can also be fluorescent lights, halogen lights, etc.

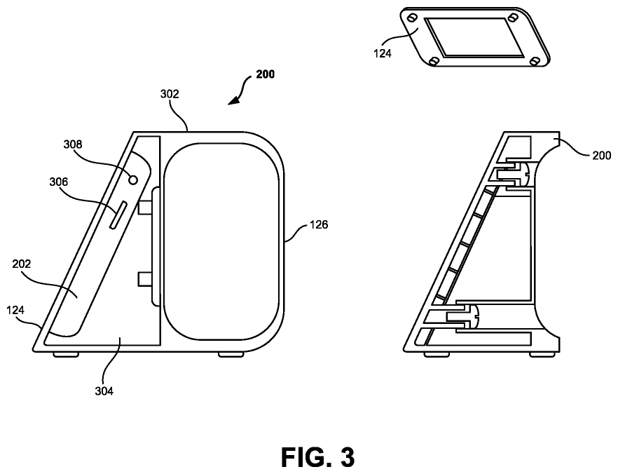

The flashlights 118 can be illuminated in several situations. For instance, the flashlights 118 can be illuminated whenever the elongated member 102 is disengaged from the docking station 200. Further, the flashlights 118 can also be illuminated whenever the user turns ON the electric toothbrush assembly 100 by pushing the power button 116 ‘ON’. In another instance, the flashlights 118 are illuminated during a reminder to the user. The reminder may include an audio/video message that motivates or reminds the user to begin the brushing event. The audio/video message can be displayed on a communication device 202 disposed in a body cavity (304, as shown in FIG. 3) of the docking station 200. In accordance with a non-limiting embodiment of the invention, the electric toothbrush assembly 100 may play a prerecorded audio/video message as the reminder, where the audio message may include a customized recording from the user's parent. The prerecorded reminder may be contemplated as any audio or musical note that motivates the user to begin a brushing event. In a preferred embodiment, the flashlights 118 are illuminated in a synchronized manner with respect to the played reminder. The flashlights 118 can also illuminate in a sequential or non-sequential manner. Further, the flashlights 118 can also produce a pleasing sound while illuminating in the sequential or non-sequential manner. Moreover, the flashlights 118 can also repetitively illuminate until the user starts the brushing event.

FIGS. 2A shows a front view and a side view of the docking station 200, which is configured to hold and/or charge the electric toothbrush assembly 100. The docking station 200 includes a body cavity (304, as shown in FIG. 3) for holding a communication device 202 and a plurality of cavities such as a first cavity 204, a second cavity 206, and a third cavity 208 disposed thereon. In an embodiment, the electric toothbrush assembly 100 (as shown in FIG. 2B) can rest inside the second cavity 206 along its distal end 103. It should, however, be noted that the electric toothbrush assembly 100 can rest inside any suitable cavity in a manner that the assembly 100 get charged while sitting within the cavity. Also, the first cavity 204 and the third cavity 208, respectively, may accommodate one or more spare brush head (120, as shown in FIG. 1A) and/or a toothpaste (122, as shown in FIGS. 1A and 1B). In another embodiment, the first cavity 204 and the third cavity 208, respectively, may be used to hold one or more spare electric toothbrush assembly (121, as shown in FIG. 1B) and/or toothpaste 122. Further, the communication device 202 may include a suitable electronic visual display device including, but not limited to, tablet, mobile phone, handheld phone, laptop, and personal digital assistant (i.e. PDA).

The docking station 200 further includes a cable 210 that can be used to connect the docking station 200 to an auxiliary device. In an exemplary embodiment, the cable 210 is used to connect the docking station 200 to a charging system (not shown). In another embodiment, the cable 210 connects the docking station 200 to a computing device, for example, a computer system, laptop, tablet, and the like.

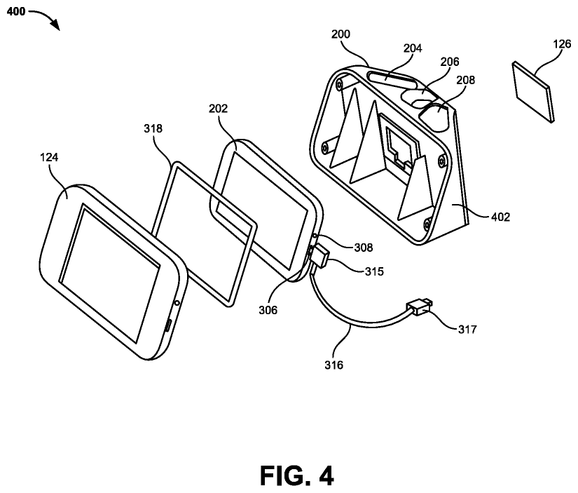

FIGS. 3 and 4 shows respectively a partially exploded side view and an exploded side view of the docking station 200. As illustrated in FIG. 3, the docking station 200 includes a body 302 forming a body cavity 304. The shape of the body cavity 304 is formed such as to accommodate the communication device 202 and other electronics components (not shown) of the docking station 200. The docking station 200 further includes a front cover 124 and a back cover 126. While the front cover 124 secures the communication device 202 when disposed on the docking station 200, the back cover 126 protects the battery (not shown) and other electronics components of the docking station 200 from external environment.

Further, the communication device 202 includes a USB connector 306 and an input jack 308. The USB connector 306 may include any suitable USB port that allows exchange of data between one or more of the communication device 202, electric toothbrush assembly 100, and any other communication device. The USB connector 306 may include a suitable USB port including, but not limited to, USB A, USB 2.0 B, USB 3.0 B, USB C, USB 2.0 micro, USB 3.0 micro, USB mini 5.

Further, a USB cable 316, as shown in FIG. 4, may connect the communication device 202 to the electric toothbrush assembly 100 (as shown in FIG. 1). The USB cable 316 includes a proximal connector 315 that connects the USB cable 316 to the communication device 202 and a distal connector 317 connecting the USB cable to an auxiliary device (not shown). Such a wired connection can allow exchange of data files between two devices. For instance, the data file(s) exchanged between the communication device 202 and an auxiliary device or the electric toothbrush assembly 100 may include an audio file, a video file, an image file and the like. In one embodiment, the image(s) captured by the camera 106, as discussed previously, is transferred to the communication device 202 via the USB cable 316.

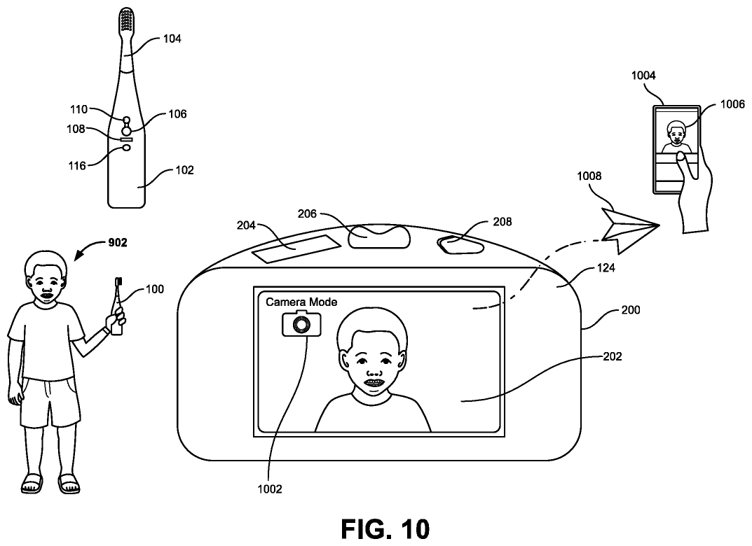

As discussed previously, the communication device 202 may include any suitable display device including, but not limited to, PDA, laptop, computer system, tablet and the like. Further, the communication device 202 may connect to one or more auxiliary device or the electric toothbrush assembly 100 using a wireless means including but not limited to, Bluetooth, zigbee, NFC, WiFi and the like. The images/video captured by the camera 106 of the electric toothbrush assembly 100 are transferred to the docking station 200 and/or other auxiliary device via a communication interface (1008, as shown in FIG. 10). The communication interface may include a wired and/or a wireless connection. The wireless connection may include, but not limited to, a Bluetooth network, a radio frequency network, a zigbee network, NFC, WiFi and the like. It should be noted that the docking station 200 may communicate with the electric toothbrush assembly 100 and/or other auxiliary devices using the same communication interface.

Further, the input jack 308 may include a headphone jack that allows the user to listen to the one or more data file(s) stored in the memory (not shown) of the communication device 202. In some embodiments, the input jack 308 may include one of a microphone jack, a stereo plug, an auxiliary input and the like.

Further, FIG. 4 illustrated an exploded view 400 of the docking station 200. The docking station includes an outer body 402 and the communication device 202. The communication device can be protected from the external environment by covering its display screen with a water proofing layer 318. In accordance with a non-limiting exemplary embodiment of the invention, the water proofing layer 318 includes a hollow rubber that seals the boundary of the display screen of the communication device 202 to avoid entry of humidity and/or air. The water proofing layer 318 can be made up of any suitable material including, but not limited to, glass, plastic, etc. such that the layer 318 protects the display screen of the communication device 202 from the external environment.

FIG. 5 shows an exploded view of the electric toothbrush assembly 100. As discussed previously, the two main components of the electric toothbrush assembly 100 include the elongated member 102 and the brush head 104. The elongated member 102 includes an outer sheath 502, a control unit 504, a chargeable battery 506, a back panel 508, flashlights 118, and the camera 106 and components holding the camera 106 on the elongated member 102.

The outer sheath 502 is configured to protect the elongated member 102 from the external environment. The outer sheath 502 includes a fitting hole 510 disposed axially along a proximal end 503 of the outer sheath 502 such that the fitting hole 510 has dimensions suitable to receive a distal end 501 of the brush head 104. The fitting hole 510 shown in FIG. 5 in the illustrated embodiment is triangular in form, however, other suitable cross-sectional shapes such as rectangular, circular, elliptical, oval, polygonal, or irregular may also be contemplated. Further, the outer sheath 502 may be made up of a plastic material including, but not limited to, polypropylene and the like. It should be noted that the outer sheath 502 may made up of any synthetic and/or natural substance. For instance, the outer sheath 502 may be made up of a biodegradable substance.

In an embodiment, a tightening member such as an adjustment plunger 511 is used to tighten the brush head 104 to the elongated member 102. Those skilled in the art may appreciate that any suitable tightening member may be used to couple the brush head 104 to the elongated member 102. Exemplary tightening members may include one or more of a nail, threaded screw, and the like.

The outer sheath 502 further includes a plurality of hollow cavities. For example, a first hollow cavity 512 is configured to receive the power button 116, a second hollow cavity 514 adapted to receive the swiveling member 108, a third hollow cavity 516 adapted to receive the camera 106, and a fourth hollow cavity 518 adapted to receive a speaker 520.

According to a non-limiting exemplary embodiment of the invention, the camera 106 includes a camera lens 516a, a camera module 516b and a coupling module 516c. The camera module 516b is mounted on the coupling module 516c. Further, the swiveling member 108 includes a first swiveling member 514a and a second swiveling member 514b such that the first swiveling member 514a fits inside the second hollow cavity 514 and the second swiveling member 514b remain coupled to the coupling module 516c.

It should be noted that the first swiveling member 514a can be pushed axially towards the coupling module 516c turning the camera 106 to the enabled state. In this state, the push of first swiveling member 514a further pushes the coupling module 516c to hit a camera trigger 522. Hitting the camera trigger 522 in turn enables the camera module 516b to click an image. It should be noted that the camera module 516b captures a single image each time the camera trigger 522 is hit. That means, the user needs to push the first swiveling member 514a each time he/she wants to capture an image. Further, the user may long press the first swiveling member 514a to initiate a video recording. To stop the video recording, the user needs to push back the first swiveling member 514a.

Those skilled in the art may understand that the camera 106 can be a visible light camera, an infrared camera, or any other camera known in the art.

In one embodiment, the control unit 504 of the electric toothbrush assembly includes a controller such as a printed circuit board (PCB). In another embodiment, the control unit 504 includes a microcontroller such as an Arduino Micro microcontroller, and the like. The control unit 504 is configured to include preprogrammed events to control the electric components of the electric toothbrush assembly 100. For example, the control unit 504 may switch the camera 106 to the enabled state to capture an image of the user, whenever the user pushes the first swiveling member 514a. In addition, the control unit 504 may switch ON the motor (not shown) to allow the rotatory movement of the brush head 106, whenever the power button 116 is turned ON by the user. It should be noted that the motor receives electric energy from the battery 506 for any such operations. To that end, the battery 506 may include a stack of rechargeable batteries that are charged by inductive or capacitive charging when the electric toothbrush assembly 100 sits in the second cavity 206 of the docking station 200. The battery 506 is held in place by battery cover 112, that is provided with an O-ring seal to further ensure that fluids do not reach the electrical components of the electric toothbrush assembly 100. Other components of the electric toothbrush assembly 100 including flashlights 118 are protected from external environment using the back panel 508.

Although not shown, the docking station 200 includes a charging means such as an induction charging unit, which provides electric charge to the electric toothbrush assembly 100 whenever the electric toothbrush assembly 100 rests inside the second cavity 206 (as shown in FIG. 2A) of the docking station 200. It should be noted that the docking station 200 may include any suitable charging means that allows transfer of electric charge to the electric toothbrush assembly 100. To that end, the charging means may include direct power supply, battery, and the like. As shown in FIG. 5, the electric toothbrush assembly 100 includes a charging hole 509 that allows connection of the electric toothbrush assembly 100 to an external power supply, including but not limited to, power supply from electric mains, portable chargers, charging from communication devices like a computer, a laptop, a tablet, a mobile phone, etc.

FIG. 6 is a schematic view of the electric toothbrush assembly 100 depicting the camera 106 along with its main components. The horizontal movement of the first swiveling member 514a inside the second hollow cavity 514 allows the opening and closing of the camera cover 110. For instance, horizontal movement of the first swiveling member 514a towards the right side of the second hollow cavity 514 allows the opening of the camera cover 110, thereby allowing the use of the camera 106 to capture an image/video (enabled state). In contrast, moving the first swiveling member 514a towards the left side closes the camera cover 110 switching the camera 106 to the disabled state. As discussed previously, the camera cover 110 should remain closed whenever the camera 106 is not in use, which protects the camera 106 from the external environment.

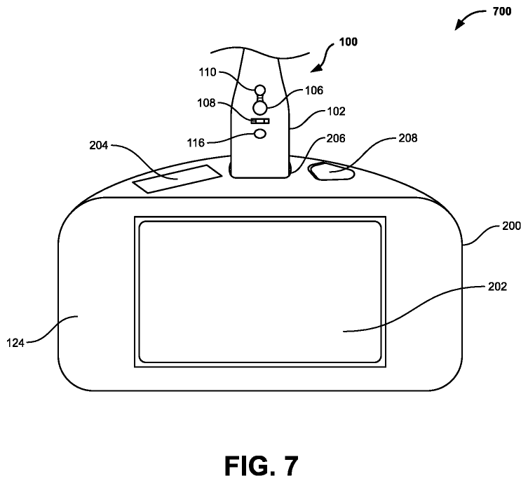

FIG. 7 shows a front view of the docking station 200 in an idle state (sleep mode) 700. The embodiment depicts the electric toothbrush assembly 100 resting inside the second cavity 206 of the docking station 200. In such embodiment, the electric toothbrush assembly 100 remains in the engaged state with the docking station 200 such that the battery 506 (as shown in FIG. 5) of the electric toothbrush assembly 100 can be charged. As discussed previously, the electric toothbrush assembly 100 can be inductively or capacitively charged by the docking station 200. To accomplish this, in an embodiment, the charging port 602 (as shown in FIG. 6B) of the elongated member 102 may include a primary coil (not shown) and the docking station 200 may include a secondary coil that inductively charges the battery 506 of the electric toothbrush assembly 100.

Typically, the communication device 202 may remain off while the docking station 200 is in the sleep mode. In such cases, the docking station 200 charges the electric toothbrush assembly 100 in relatively shorter time, as compared to a scenario when a user may switch on the user display of the communication device 202 and the docking station 200 charges the electric toothbrush assembly 100.

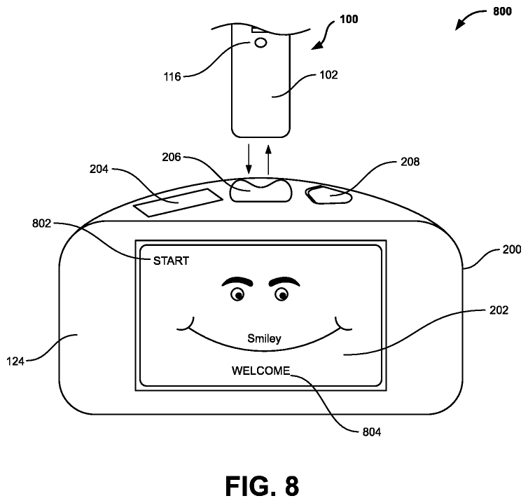

FIG. 8 shows the electric toothbrush assembly 100 in a disengaged position 800 from the docking station 200. As discussed previously, the electric toothbrush assembly 100 may switch to the disengaged position whenever a user removes the elongated member 102 out of the docking station 200. Typically, the elongated member 102 remains in the disengaged position whenever the electric toothbrush assembly 100 is held by the user for one or more brushing events.

Disengaging the elongated member 102 from the docking station 200 may initiate a start mode 802 on the docking station 200. In the start mode 802, a first message 804 is displayed on the communication device 202. In FIG. 8, the first message 804 is shown as a combination of a text “WELCOME” and an image. The first message 804 may include a suitable multimedia file that may provide welcome note to the user or motivate the user for oral hygiene. In some embodiments, the first message 804 may include one or more of an image, an audio, a video and a combination thereof. In an exemplary embodiment, the first message 804 may include a homepage of a multimedia game that motivates the user to perform an effective brushing event.

Disengaging of the elongated member 102 from the docking station 200 may also illuminate the flashlights 118. The flashlights 118 can illuminate in a sequential or non-sequential manner. Further, the flashlights 118 can also produce a pleasing sound while illuminating in the sequential or non-sequential manner. Moreover, the flashlights 118 can also repetitively illuminate until the user starts the brushing event.

The effective brushing is accomplished when the brush head 104 moves back and forth such that the bristles 105 clean the inner surface, outer surface and chewing surfaces of the teeth to avoid formation of plaque, dental cavity and other oral complications. In a preferred embodiment, effective brushing is achieved when the user brushes his/her teeth for a predetermined time interval to meet a predetermined condition. In an exemplary embodiment, the predetermined condition can be met when a user brushes his/her teeth for a time interval of at least 2 minutes that maintains the overall oral hygiene and avoids formation of any oral complications. Alternatively, the predetermined condition may also comprise brushing on all sides of teeth. Further, the predetermined condition may comprise cleaning tongue in addition to teeth. However, it should be noted that the predetermined condition may include any suitable brushing time, and other criteria that allows effective brushing to avoid cavities and other oral complications.

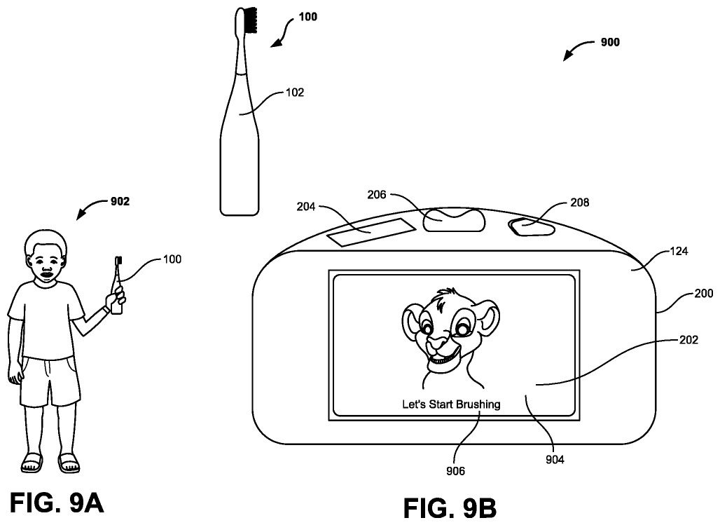

FIG. 9A illustrates a user 902 holding the electric toothbrush assembly 100 in a disengaged position FIG. 9B illustrates the initiation of a brushing mode 904 on the docking station 200 according to an embodiment 900 of the invention.

The brushing mode 904 teaches/motivates the user 902 to start with the brushing event. Such mode can also be termed as a “progressive learning mode”, as the communication device 202 displays a message 906 that provides required learning to the user 902 to perform effective brushing. The learning can be provided to the user 902 in form of text instructions, a video displaying effective brushing way, an instruction audio or in any other form which motivates and teaches the user 902 to brush teeth in an effective manner. In the brushing mode 904, the flashlights 118 on the electric toothbrush assembly 100 may also be illuminated and synchronized with the message 906 displayed on the communication device 202. The flashlights 118 can also illuminate in a sequential or non-sequential manner. Further, the flashlights 118 can also produce a pleasing sound while illuminating in the sequential or non-sequential manner. Such a synchronization of the message 906 and flashlights 118 may engage and/or motivate the user to start with the brushing event.

In some embodiments, the message 906 may include one or more of an image, an audio, a video and a combination thereof. For instance, the message 906 may include a suitable multimedia file that provides brushing instructions to the user 902. In another exemplary embodiment, the message 906 may include a multimedia game that provides instructions to the user and aids in achieving the effective brushing. It should be noted that the effective brushing is accomplished when the brush head 104 moves back and forth such that the bristles 105 clean the inner surface, outer surface and chewing surfaces of the teeth to avoid formation of plaque, dental cavity and other oral complications.

FIG. 10 is a schematic view depicting a camera mode 1002 of the docking station 200. It should be noted that the camera mode 1002 is initiated only when the user 902 completes the brushing event (as shown in FIG. 9) meeting the predetermined condition of effective brushing.

In the camera mode 1002, the docking station 200 communicates with a remote communication device 1004 (for example, a second communication device) to share a second message 1006. In an embodiment, the second message 1006 includes a selfie image of the user 902, which is shared with the remote communication device 1004 only upon meeting the predetermined condition (i.e. brushing event for a predetermined time interval of at least 2 minutes). The second message 1006 may further include any suitable message that depicts successful completion of the brushing event meeting the predetermined condition. In an alternative embodiment, the second message 1006 can be a video image of the user 902 showing his/her cleaned teeth. Further, the second message 1006 can also be an audio recording of user's 902 voice confirming that he/she has cleaned the teeth. The second message 1006 can also be a combination of a text, video and audio. The electric toothbrush assembly 100 can share the second message 1006 with the remote communication device 1004 directly via the communication interface (not shown in FIG. 10) through a communication interface (not shown in FIG. 10). Alternatively, the electric toothbrush assembly 100 can send the second message 1006 to the communication device 202. The communication device, then, sends the second message 1006 to the remote communication device 1004 through a communication interface 1008, as illustrated in FIG. 10. The communication interface 1008 and the communication interface between the electric toothbrush assembly 100 and the remote communication device 1004 may include a wired or a wireless communication interface. Exemplary communication interface includes a Bluetooth connection, a Zigbee connection, an NFC connection and a WiFi connection. It should, however, be noted that any other communication interface can be used for transmitting the second message 1006.

Further, the remote communication device 1004 may include, but not limited to, a mobile phone, a tablet, a laptop, a computer system, a PDA and the like. In some embodiments, the remote communication device 1004 includes a mobile phone used by a parent of the user 902. On receiving the second message 1006, the parent may in return share a message that can be displayed on the communication device 202. The message may include a suitable multimedia message. For instance, the parent may share a thumbnail ‘thumbs up’ to congratulate and motivate the user 902 to keep good habit of effective brushing.

Thereby the above invention creates a fun environment to motivate and assist the user 902 to learn the effective brushing technique. In addition, the communication with remote communication device 1004 allows a parent or any other known individual to communicate and help the user 902 to develop a regular dental hygiene regime to decrease cavities, gum disease, and other dental complications from lack of brushing.

Other embodiments of the invention will be apparent to those skilled in the art from consideration of the specification and practice of the invention disclosed herein. It is intended that the specification examples be considered as exemplary only.

What is claimed is:

1. An electric toothbrush assembly comprising:
a brush head;
an elongated member having a proximal end configured to couple to the brush head and a distal end configured to engage with a docking station assembly, wherein the elongated member is configured to switch between an engaged position and a disengaged position with the docking station assembly;
a camera configured to capture image of a user, wherein the camera is configured to switch between an enabled state and a disabled state; and
a communication interface, wherein the communication interface enables communication between the elongated member and one or more communication devices,
wherein a first message is displayed on a user display of a first communication device of the one or more communication devices when the state of elongated member changes from engaged position to disengaged position, and
wherein a second message is sent to a second communication device of the one or more communication devices when a predetermined condition is satisfied.

2. The electric toothbrush assembly of claim 1, wherein the first message includes a multimedia file providing brushing instructions to the user.

3. The electric toothbrush assembly of claim 2, wherein the multimedia file comprises one or more of an image, an audio and a video.

4. The electric toothbrush assembly of claim 1, wherein the first message is a multimedia game that aid in achieving effective brushing.

5. The electric toothbrush assembly of claim 1, wherein the second message comprises one or more of an image, an audio and a video of the user captured through the camera.

6. The electric toothbrush assembly of claim 1, wherein the camera captures one or more of an image, an audio and a video of the user in the enabled state.

7. The electric toothbrush assembly of claim 1, wherein the predetermined condition is a brushing event for a predetermined time interval.

8. The electric toothbrush assembly of claim 1, wherein the one or more communication devices comprise a mobile phone, a computer, a laptop and a tablet.

9. The electric toothbrush assembly of claim 1, wherein in the engaged position the docking station charges the electric toothbrush assembly.

10. The electric toothbrush assembly of claim 1, wherein in the disengaged position the elongated member is removed out of the docking station assembly.

11. The electric toothbrush assembly of claim 1, wherein the elongated member remains in the disengaged position whenever the electric toothbrush assembly is in use for oral hygiene.

12. The electric toothbrush assembly of claim 1 further includes a plurality of flash lights.

13. The electric toothbrush assembly of claim 12, wherein the flash lights include light emitting diodes.

14. The electric toothbrush assembly of claim 1 further includes a control unit configured to assess a brushing event based on the predetermined condition.

15. The electric toothbrush assembly of claim 14, wherein the control unit detects the effectiveness of the brushing event based on one or more of movement direction, speed and time of the brush head.

16. The electric toothbrush assembly of claim 1 further includes a memory device.

17. The electric toothbrush assembly of claim 16, wherein the memory device is configured to store data related to one or more brushing events.

18. The electric toothbrush assembly of claim 16, wherein the memory device stores one or more files related to guiding the user on effective brushing techniques.

19. The electric toothbrush assembly of claim 18, wherein the file includes a pre-recorded brushing game.

20. The electric toothbrush assembly of claim 1, wherein the elongated member further includes a chargeable battery.

21. The electric toothbrush assembly of claim 1, wherein the elongated member further includes a push button configured to switch the electric toothbrush assembly between an ON state and an OFF state.

22. The electric toothbrush assembly of claim 1, wherein the elongated member further includes a charging port to connect and charge the electric toothbrush assembly through an electric power supply.

23. The electric toothbrush assembly of claim 1, wherein the communication interface is a wireless connection.

24. The electric toothbrush assembly of claim 23, wherein the wireless connection comprises one or more of a Bluetooth connection, Zigbee connection, an NFC connection and a WiFi connection.

25. The electric toothbrush assembly of claim 1, wherein the communication interface is a wired connection.

26. The electric toothbrush assembly of claim 1, wherein the brush head is removably coupled to the proximal end of the elongated member.

27. The electric toothbrush assembly of claim 1 further includes a motor configured to provide movement to the brush head.

28. The electric toothbrush assembly of claim 27, wherein said movement enables the user to use the electric toothbrush assembly for oral hygiene during the one or more brushing events.
